(19) United States
(12) Patent Application Publication
(10) Pub. No.: US 2008/0046092 A1
(43) Pub. Date: Feb. 21, 2008

(54) Coil embolization device with stretch resistance fiber

(76) Inventors: Richard Champion Davis, Plantation, FL (US); Donald K. Jones, Dripping Springs, TX (US); Juan A. Lorenzo, Davie, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/506,083

(22) Filed: Aug. 17, 2006

(51) Int. Cl. A61F 2/02

(52) U.S. Cl. 623/23.72

(57) ABSTRACT

A vasoocclusive embolic device deployment system for use in the placement of an embolic coil at a treatment site within a vessel. The embolic coil includes an elongated stretch resistant fiber having its distal end attached to the distal end of the embolic coil. The stretch resistant fiber extends cylindrically around the outer surface of the coil and the proximal end of the stretch resistant fiber is attached to the proximal end of the coil. The stretch resistant fiber preferably takes the form of a loosely wound helical coil and may also be attached to the embolic coil at additional points between the proximal and distal ends of the embolic coil. Additionally, a headpiece is mounted at the proximal end of the embolic coil for coupling the coil to a coil deployment device.

### BACKGROUND OF INVENTION

1. Field of the Invention

The present invention relates to a medical device designed for implantation within a vessel of the body, and more particularly, to a stretch resistant vasoocclusive coil for the treatment of aneurysms. The vasoocclusive coil is particularly suited for use in cases where it may be necessary to reposition the coil once the coil has been initially placed within an aneurysm.

2. Description of the Prior Art

For many years, vasoocclusive devices have been used to occlude blood vessels at specific treatment locations. These devices take many different forms including helically wound coils, coils wound within coils, or other such coil configurations. Examples of various coil configurations are disclosed in U.S. Pat. No. 5,334,210, entitled “Vascular Occlusion Assembly;” and U.S. Pat. No. 5,382,259, entitled, “Vasoocclusion Coil with Attached Tubular Woven or Braided Fibrous Covering.” Embolic coils are generally formed of a radiopaque metallic material, such as platinum, gold, tungsten, or an alloy of these metals. Often, several coils are placed at a given location to occlude, or partially occlude, the flow of blood through the vessel, or aneurysm, by promoting thrombus formation at the particular location.

Flexible catheters have been used to place various devices or medications within the vasculature of the human body. Such devices or medications include dilation balloons, radiopaque fluids, liquid medications, and various types of occlusion devices such as balloons and embolic coils. Examples of such catheter-based devices are disclosed in U.S. Pat. No. 5,108,407, entitled “Method and Apparatus for Placement of an Embolic Coil;” and U.S. Pat. No. 5,122,136, entitled “Endovascular Electrolytically Detachable Guidewire Tip for the Electroformation of Thrombus in Arteries, Veins, Aneurysms, Vascular Malformations and Arteriovenous Fistulas.” These patents disclose catheter-based devices designed to deliver embolic coils to a predetermined site within a vessel of the human body in order to treat aneurysms, or alternatively, to occlude a blood vessel at a particular location.

Additionally, embolic coils have been placed within the distal end of a catheter, such that when the distal end of the catheter is properly positioned, the coil may then be pushed out of the end of the catheter with a pusher member to release the coil at the predetermined site within the vessel. This procedure for placement of the embolic coil is conducted under fluoroscopic visualization, such that the movement of a coil through the vasculature of the body may be monitored, and the coil may be placed in the desired location.

To prevent stretching of the embolic device, especially during post-deployment retrieval, or repositioning during delivery, embolic devices often take the form of an embolic coil having a lumen extending therethrough and a stretch resistant member extending through the lumen. In one embodiment, the stretch resistant member takes the form of a fiber which is attached to the proximal and distal ends of the coil. In another embodiment, the stretch resistant member is fixedly attached to the distal end of the embolic coil, is extended through the lumen of the coil, and is detachably connected to a proximal end of an elongated pusher member. The connection between the pusher member and the coil may be severed by application of heat to the stretch resistant member, typically formed of a thermoplastic material. Such a device is disclosed in U.S. Patent Publication No. 2004/0034363, entitled “Stretch Resistant Therapeutic Device.” Another variation of a stretch resistant embolic device includes a helically wound outer coil with a stretch resistant member extending therethrough. In order to prevent stretching during movement of the coil, the stretch resistant member is fixedly attached to the coil in at least two locations, such as the proximal end and the distal end. The coil may take on a secondary shape when it is released from the delivery device. Such a device is disclosed in U.S. Pat. No. 5,853,418, entitled “Stretch Resistant Vasoocclusive Coils (II).”

Yet another embodiment of a stretch resistant coil includes a stretch resistant member, such as a fiber, which extends through at least a portion of a primary coil having proximal and distal ends. The stretch resistant member is attached to the primary coil at two axially separated locations to prevent or minimize axial stretching of the coil. One of these attachment locations is created with an anchor assembly disposed within the lumen of the coil. The anchor assembly takes the form of a coil that is incorporated into the windings of the primary coil. Such a device is disclosed in US Patent Publication No. U.S. 2004/0002733, entitled “Integrated Anchor Coil in Stretch-Resistant Vaso-occlusive Coils.”

Still another embodiment of a stretch resistant coil and delivery system takes the form of an interlocking coupling between a pusher member and a thin wire affixed to an embolic coil. The thin wire may be affixed to a distal, intermediate or proximal location on the coil and includes a ball shaped member fixedly attached to the proximal end of the wire. In order to position the coil at the treatment site, a pusher member with a ball member affixed to its distal end releasably interlocks with the ball member at the proximal end of the stretch resistant member. Such a device is disclosed in U.S. Pat. No. 5,304,195, entitled “Detachable Pusher-Vasoocclusive Coil Assembly with Interlocking Coupling.”

Even another embodiment of a stretch resistant embolic coil includes a coil with proximal and distal ends reinforced with a stretch resistant member having proximal and distal ends extending therethrough. The distal end of the stretch resistant member is fixedly attached at the distal end of the coil, and the proximal end of the stretch resistant member is detachably mounted on an elongated pusher member at its distal end. Such a device is disclosed in U.S. Patent Publication No. 2005/0043755, entitled, “Vasoocclusive Coil with Enhanced Therapeutic Strand Structure.”

Yet another embodiment of a stretch resistant embolic coil includes a wire that is wrapped with a polymer and is helically wound. A stretch resistant member may extend though the lumen of the coil and is attached to at least two points on the coil. Such a device is disclosed in U.S. Pat. No. 6,280,457, entitled, “Polymer Covered Vasoocclusive Devices and Methods of Producing Such Devices.”

Another embodiment of an embolic device with multiple elements is a three element coaxial vaso-occlusive device which includes an inner core element, such as a coil, a non-metallic expansile intermediate element, and a non-expansile generally tubular outer element covering at least a part of the intermediate element, and having at least one gap therein. When the embolic device is deployed and the intermediate element expands through at least one gap in the outer element, creating an undulating outer surface. Such a device is disclosed in U.S. Patent Publication No. 2004/0034363 entitled, “Three Element Coaxial Vaso-occlusive Device.”

### SUMMARY OF THE INVENTION

The present invention is directed toward a vasoocclusive device deployment system for use in placing an embolic device at a preselected site within a blood vessel or aneurysm. In accordance with an aspect of the present invention, the deployment system includes an elongated flexible delivery catheter and an elongated flexible deployment catheter slidably disposed within the lumen of the delivery catheter. Also included is a generally cylindrical embolic coil, which preferably is helically wound. A stretch resistant fiber is attached to the proximal end of the embolic coil and extends cylindrically around the outer surface of the embolic coil. The distal end of the fiber is attached to the distal end of the embolic coil. Preferably, the stretch resistant fiber takes the form of a loosely spaced helically wound embolic coil. Additionally, the stretch resistant fiber may be attached to the embolic coil at additional points along the length of the coil. Also, the embolic device generally takes on a secondary shape after deployment.

In accordance with another aspect of the present invention, a headpiece is mounted on the proximal end of the embolic coil and is disposed in fluid tight engagement within the lumen of the distal section of the deployment catheter. A source of fluid pressure is coupled to the proximal section of the deployment catheter for applying a fluid pressure to the headpiece to thereby release the embolic coil from the deployment catheter. The distal section of the deployment catheter is preferably formed of a material which exhibits the characteristic that when fluid pressure is applied to the lumen of the deployment catheter the distal section of the deployment catheter expands outward to release the headpiece.

In accordance with yet another aspect of the present invention, an embolic device includes a helically wound embolic coil. A stretch resistant fiber is bonded to the proximal end of the embolic coil and extends cylindrically around the outer surface of the embolic coil. The distal end of the fiber is bonded to the distal end of the embolic coil. Preferably, the stretch resistant fiber takes the form of a loosely spaced helically wound embolic coil. Additionally, the stretch resistant fiber may be attached to the embolic coil at additional points along the length of the coil. A headpiece is mounted on the proximal end of the embolic coil in order to couple the embolic device to a deployment system.

In accordance with still another aspect of the present invention, an embolic device includes a generally helically wound embolic coil. A stretch resistant fiber takes the form of a loosely spaced helically wound coil and is bonded to the proximal section of the embolic coil. The stretch resistant fiber extends cylindrically around the outer surface of the embolic coil and the distal end is bonded to the distal section of the embolic coil. Additionally, the stretch resistant fiber may be attached to the embolic coil at additional points along the length of the coil. A headpiece is mounted on the proximal end of the embolic coil in order to couple the embolic device to a deployment system.

### BRIEF DESCRIPTION OF THE DRAWINGS

FIG. 1 is an enlarged, partially sectional view of one embodiment of a stretch resistant vasoocclusive device deployment system in accordance with the present invention; and,

FIG. 2 is an enlarged view of the stretch resistant embolic device shown in FIG. 1.

### DESCRIPTION OF THE PREFERRED EMBODIMENT

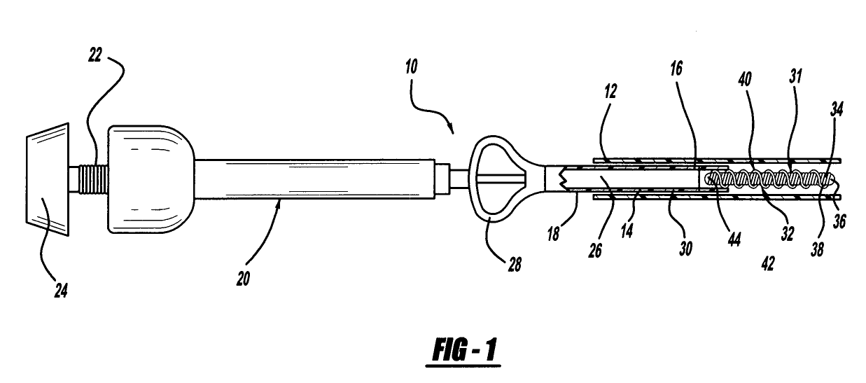

FIG. 1 generally illustrates one embodiment of a stretch resistant vasoocclusive device deployment system 10 of the present invention, including an elongated flexible delivery catheter 12 having an elongated flexible deployment catheter 14 slidably disposed within the lumen 16 of the delivery catheter 12. A stretch resistant embolic device 31 is disposed within the lumen 26 of the distal section 30 of the deployment catheter 14. A source of fluid pressure which preferably takes the form of a syringe 20 is coupled to the proximal section 18 of the deployment catheter 14. The syringe 20 includes a threaded piston 22 which is controlled by a handle 24 to thereby infuse fluid into the lumen 26 of the deployment catheter 14. As illustrated, the proximal section 18 of the deployment catheter 14 includes a winged hub 28 which aides in the insertion of the deployment catheter into the vasculature of the body.

The stretch resistant embolic device 31 is disposed within the lumen 26 of the distal section 30 of the deployment catheter 14. The stretch resistant embolic device 31 includes a cylindrical embolic coil 32 having an atraumatic distal bead 36 bonded to the distal end of the coil 32. Also included is a stretch resistant fiber 40 which has a proximal end 48 bonded to the proximal end of the coil 32 and extends cylindrically around the outer surface of the embolic coil 32. The distal end 46 of the stretch resistant fiber 40 is attached to the distal end 38 of the coil 32. Additionally, a headpiece 44 is mounted on the proximal end 42 of the coil 32 which, in turn, is disposed in fluid tight engagement within the lumen 26 of the distal section 30 of the deployment catheter 14.

When the embolic coil 32 is at the desired treatment site, the handle 24 is manipulated to advance the threaded piston 22, which thereby infuses fluid into the lumen 26 of the deployment catheter 14. The fluid is advanced through the lumen 26 of the deployment catheter 14 and pressure is applied to the proximal end of the headpiece 44 to thereby displace it from its position within the distal section 30 of the deployment catheter 14.

If desired, the distal section 30 of the deployment catheter 14 may be formed from a material having a different durometer from that used to form the proximal section 18. For example, the proximal section 18 of the deployment catheter 14 may be formed of Pebax material having a durometer in the range of about 62 D to 75 D. The proximal section 18 will then be sufficiently flexible to traverse the vasculature of the human body, but also sufficiently rigid such that when a fluid pressure of approximately 300 psi is applied to the interior of this end of the deployment catheter there is little, if any, radial expansion of the walls of this section of the deployment catheter. In contrast, the distal section 30 of the deployment catheter 14 may be formed from a polymer material with a relatively low durometer. The distal section 30 of the deployment catheter 14 is preferably formed from a block copolymer, such as Pebax, having a durometer in a range of 25 D to 55 D with a preferred durometer of 40 D.

The lower durometer material used to form the distal section 30 of the deployment catheter 14 exhibits the characteristic that when a fluid pressure of approximately 300 psi is applied to the interior, the walls of the distal section 30 expand radially, somewhat similar to the action of a balloon inflating, to thereby release the headpiece 44 of the embolic coil 32.

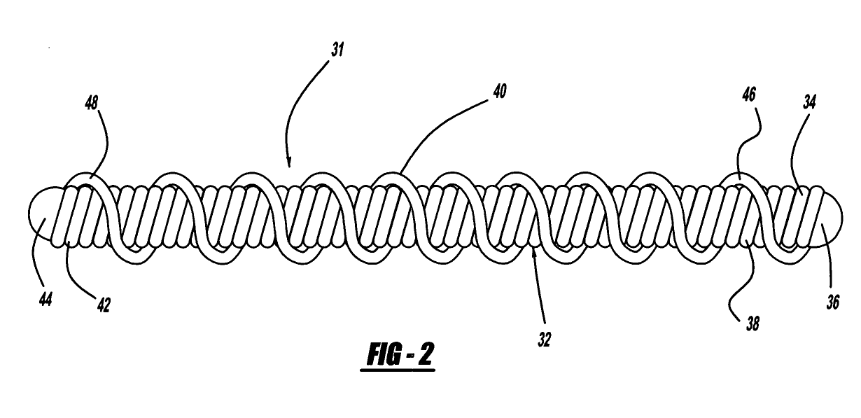

FIG. 2 illustrates in greater detail the stretch resistant embolic device 31 for placement at a treatment site. The stretch resistant embolic device 31 includes the cylindrical embolic coil 32 having the atraumatic distal bead 36 bonded to the distal end 38 of the coil 32. Also included is a stretch resistant fiber 40 which is attached to the proximal end 42 of the coil 32 and extends cylindrically around the outer surface of the coil 32. The distal end 46 of the stretch resistant fiber 40 is attached to the distal end 38 of the coil 32. The stretch resistant fiber 40 may be attached to the embolic coil at additional points between the distal end 38 and the proximal end 42 of the embolic coil 32. The headpiece 44 is mounted on the proximal end 42 of the coil 32 and couples the stretch resistant embolic device 31 to the deployment system 10.

More particularly, the cylindrical embolic coil 32 is preferably formed of helical turns 34 and is constituted from a platinum tungsten alloy. The atraumatic distal bead 36 has a generally hemispherical shape and is formed of a plasma bead or a solder weld. The stretch resistant fiber 40 preferably takes the form of a coil having helical turns and being constituted from a platinum tungsten alloy. In order to impart stretch resistance, the stretch resistant fiber 40 taking the form of a coil has fewer helical turns than the cylindrical embolic coil 32 and is therefore of a length that is shorter than that of the cylindrical embolic coil 32. Alternately, the fiber 40 may take the form of a nitinol wire or polymer braid or filament. Further, the stretch resistant fiber 40 in the form of a coil may have a diameter that differs from that of the cylindrical embolic coil. Additionally, the fiber 40 may be welded to the points of attachment at the proximal end 42 and distal end 38 of the cylindrical embolic coil 32 and at any additional points along the length of the cylindrical embolic coil 32. The headpiece 44 is formed from a metallic or polymeric material and preferably takes a generally hemispherical shape.

During placement of the cylindrical embolic coil 32 at the treatment site, fluid pressure is applied to the headpiece 44 to dislodge the headpiece and thereby the stretch resistant embolic device 31 from the deployment system 10. An important advantage of the present invention is that if it is determined that the embolic device 31 is improperly positioned the embolic device may then be withdrawn from that location and placed at another location, or even removed from the body altogether. The stretch resistant fiber 40 facilitates repositioning of the embolic device 31 because it prevents the cylindrical coil 32 from stretching when it is pulled proximally from the improper position. Once the embolic device 31 is released at the appropriate location, the stretch resistant fiber 40 has a limited mobility, which in part dictates a secondary shape for the embolic device 31 after it is released and no longer constrained by the deployment catheter.

Another important advantage of the present invention is that the stretch resistant fiber 40, taking the form of a coil, extends around the outer surface of the cylindrical embolic coil 32 and thus provides stretch resistance without compromising the flexibility of the device by adding bulk. The flexibility of the device is further maintained by having two different diameters and separate mobility for the cylindrical embolic coil 32 and the stretch resistant fiber 40. The two different diameters and separate mobility of the cylindrical embolic coil 32 and the stretch resistant fiber 40 also allows the stretch resistant fiber to better fit within the interstices between subsequently deployed devices.

As is apparent, there are numerous modifications of the preferred embodiment described above which will be readily apparent to one skilled in the art, such as many variations and modifications of the embolic coil including numerous coil winding configurations. There are also variations in the materials used to form the various components. Additionally, the diameters of the embolic coil and the stretch resistant fiber can be varied or can be the same, and the frequency of attachment between the embolic coil and the stretch resistant fiber can be varied to increase or decrease the stretch resistance. These modifications would be apparent to those having ordinary skill in the art to which this invention relates and are intended to be within the scope of the claims which follow.

That which is claimed is:

1. A vasoocclusive embolic device deployment system for use in placing an embolic device at a preselected site within a vessel comprising:
an elongated flexible delivery catheter having proximal and distal sections and a lumen extending therethrough;
an elongated flexible deployment catheter having proximal and distal sections and a lumen extending therethrough and being slidably disposed within the lumen of the elongated flexible delivery catheter;
a cylindrical embolic coil having proximal and distal ends and an outer surface;
a stretch resistant fiber having proximal and distal ends, the proximal end of the fiber is attached to the proximal end of the embolic coil, the fiber then extends cylindrically around the outer surface of the embolic coil and the distal end of the fiber is attached to the distal end of the embolic coil;
a headpiece is mounted on the proximal end of the coil and is disposed in a fluid tight engagement within the lumen of the distal section of the deployment catheter; and,
a source of fluid pressure is coupled to the proximal section of the deployment catheter for applying a fluid pressure to thereby release the embolic coil from the deployment catheter.

2. A vasoocclusive embolic device deployment system as defined in claim 1, wherein the stretch resistant fiber takes the form of a loosely spaced helically wound embolic coil.

3. A vasoocclusive embolic device deployment system as defined in claim 1, wherein the embolic device formed by the embolic coil and the stretch resistant fiber takes on a secondary shape after release from the deployment catheter.

4. A vasoocclusive embolic device deployment system as defined in claim 3, wherein the embolic coil comprises a helically wound embolic coil.

5. A vasoocclusive embolic device deployment system as defined in claim 1, wherein the stretch resistant fiber is attached to the embolic coil at additional points between the proximal and distal ends of the embolic coil.

6. A vasoocclusive embolic device deployment system as defined in claim 1, wherein the distal section of the deployment catheter is formed of a material which exhibits the characteristic that when fluid pressure is applied to the lumen of the deployment catheter said distal section of the deployment catheter expands outward to release the headpiece.

7. A vasoocclusive embolic device for use in placement at a treatment site within a vessel comprising:
a cylindrical embolic coil having proximal and distal ends and an outer surface;
a stretch resistant fiber having proximal and distal ends, the proximal end of the fiber is attached to the proximal end of the embolic coil and extends cylindrically around the outer surface of the embolic coil, and the distal end of the fiber is attached to the distal end of the embolic coil; and,
a headpiece is mounted on the proximal end of the embolic coil which serves to couple the embolic device to a coil deployment system.

8. A vasoocclusive embolic device as defined in claim 7, wherein the stretch resistant fiber takes the form of a loosely spaced helically wound embolic coil.

9. A vasoocclusive embolic device as defined in claim 7, wherein the embolic device formed by the embolic coil and the stretch resistant fiber take on a secondary shape after release from the coil deployment system.

10. A vasoocclusive embolic device as defined in claim 8, wherein the embolic coil comprises a helically wound embolic coil.

11. A vasoocclusive embolic device as defined in claim 10, wherein the stretch resistant fiber is attached to the embolic coil at additional points between the proximal and distal ends of the embolic coil.

12. A vasoocclusive embolic device for use in placement at a treatment site within a vessel comprising:
a cylindrical embolic coil having proximal and distal sections and an outer surface;
a stretch resistant fiber which takes the form of a loosely spaced helically wound coil having proximal and distal ends, the proximal end of the fiber is attached to the proximal section of the embolic coil, the fiber extends cylindrically around the outer surface of the embolic coil and the distal end of the fiber is attached to the distal section of the embolic coil; and,
a headpiece is mounted on the proximal section of the embolic coil and serves to couple the embolic device to a coil deployment system.

13. A vasoocclusive embolic device as defined in claim 12, wherein the embolic coil and the stretch resistant fiber take on a secondary shape after release from the coil deployment system.

14. A vasoocclusive embolic device as defined in claim 12, wherein the cylindrical embolic coil comprises a helically wound embolic coil.

15. A vasoocclusive embolic device as defined in claim 12, wherein the stretch resistant fiber is attached to the embolic coil at additional points between the proximal and distal sections of the embolic coil.
